(19) United States
(12) Patent Application Publication
(10) Pub. No.: US 2010/0041612 A1
(43) Pub. Date: Feb. 18, 2010

(54) Fragments of the Glucagon-Like Peptide-1 and Uses Thereof

(76) Inventor: Martin Beinborn, Newton, MA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 11/991,661

(22) PCT Filed: Sep. 8, 2006
(86) PCT No.: PCT/US06/35009

§ 371 Date: Oct. 6, 2009

Related U.S. Application Data
(60) Provisional application No. 60/715,098, filed on Sep. 8, 2005.

(51) Int. Cl. A61K 38/16; C07K 7/06; C07K 7/08; C07K 14/00; A61K 38/08; A61K 38/10; A61P 3/10; C07K 16/18

(52) U.S. Cl. 514/13; 530/327; 530/328; 530/326; 514/15; 514/14; 530/387.1; 530/388.1; 530/389.1

(57) ABSTRACT

GLP-1 fragments and derivatives thereof are able to interact with and activate the GLP-1 receptor GLP-1 fragments and derivatives thereof are used either alone or in combination with other pharmaceuticals in the treatment of type 1 and type 2 diabetes and other pathologies that are known to benefit from activation of the glucagon-like peptide-1 receptor.

### FIELD OF THE INVENTION

The present invention provides peptide fragments and derivatives thereof and methods of using them for treating type 1 and type 2 diabetes mellitus and other conditions.

### BACKGROUND OF THE INVENTION

Type 2 diabetes (non-immune, adult-onset) is a rising worldwide epidemic that currently affects 17 million Americans. This disease involves a dysregulation of blood glucose levels, and leads to major complications including heart disease, stroke, kidney failure, blindness, and loss of limbs. Medical therapy of diabetes must aim at continuously reducing blood glucose to normal levels, since the risk of complications cumulatively increases with even short episodes of hyperglycemia. At the same time, drug-induced episodes of hypoglycemia must also be avoided since these may lead to loss of consciousness and even death. Major efforts in the pharmaceutical industry are focused on developing novel drugs for the treatment of diabetes, which ideally will keep blood glucose within the normal range while minimizing the risk of hypoglycemia. One of the most promising strategies toward this goal is based on mimicking the actions of glucagon-like peptide-1 (GLP-1, 7-36 amide), an endogenous hormone that plays an important physiological role in maintaining blood glucose homeostasis (Kieffer T J, Habener J F 1999 Endocr Rev 20:876-913, Drucker D J 1998 Diabetes 47:159-69).

GLP-1 is produced by L-cells primarily localized in the ileal/colonic mucosa. Following food ingestion, this peptide is secreted into the circulation and acts on multiple target tissues to attenuate the postprandial increase in blood glucose levels. In the endocrine pancreas, GLP-1 enhances glucose-induced insulin secretion as well as stimulates the growth of insulin-producing beta cells (Drucker D J 2003 Mol Endocrinol 17:161-71, Urusova I A, et al. 2004 Trends Endocrinol Metab 15:27-33). Thus, synthetic GLP-1 based drugs may not only correct blood glucose levels, but may be able to prevent the further progression of beta cell deterioration if applied at the early stages of diabetes. GLP-1 also has additional peripheral and central functions, including delay of gastric emptying, protection of heart cells and induction of satiety (Holst JJ 2002 Diabetes Metab Res Rev 18:430-41, Bose A K, et al. 2005 Diabetes 54:146-51). These accessory functions may provide a rationale for using GLP-1-based treatments in the context of congestive heart failure and obesity, conditions that are often associated with diabetes.

GLP-1 acts via a secretin-type (class B) G-protein-coupled receptor (GPCR) that triggers cAMP production as the primary signal transduction pathway (Mayo K E, et al. 2003 International Union of Pharmacology. XXXV. The glucagon receptor family. Pharmacol Rev 55:167-94). The GLP-1R, like other class B receptors, is endogenously activated by a relatively large peptide agonist. Activation of this receptor on insulin-producing beta cells in the pancreas leads to an amplification of glucose-induced insulin secretion. It is of note that this effect of GLP-1 is contingent on abnormally high blood glucose, i.e. is barely detectable in normoglycemic people. Limitation of GLP-1's effect to conditions where blood glucose is too high is a very desirable feature for developing drugs based on the endogenous peptide. Other diabetes treatments, including injectable insulin and sulfonylureas, can cause excessive reduction of blood glucose if not perfectly dosed and timed. This risk is much reduced with application of GLP-1 given its mechanism of action. Furthermore, conventional drug treatment of diabetes typically induces some weight gain, which in part counteracts the beneficial effects of these therapies. It is of note that GLP-1, given its satiety-inducing properties (see above), tends to reduce rather than increase body weight.

In view of the exceptional promise that GLP-1 based drugs hold as a treatment for diabetes and potentially obesity, derivatives of this peptide are currently being developed for clinical use. GLP-1 as such cannot be therapeutically applied since the native peptide is quickly degraded by enzymatic digestion in the blood stream. A range of synthetic GLP-1 derivatives have therefore been designed to increase metabolic stability in vivo (Holz G G, Chepurny O G 2003 Curr Med Chem 10:2471-84, Knudsen L B 2004 J Med Chem 47:4128-34-10, Meier J J, Nauck M A 2004 Curr Opin Investig Drugs 5:402-10). In addition, a lizard peptide with prolonged biological half life, exendin-4, has been identified (Raufinan J P 1996 Regul Pept 61:1-18, Giannoukakis N 2003 Curr Opin Investig Drugs 4:459-65) which despite limited sequence homology to GLP-1 acts as a potent full agonist of the GLP-1 receptor. A remaining disadvantage of utilizing either GLP-1 analogues or exendin-4 clinically is that these relatively large 30-39 amino acid molecules require parenteral application.

The present invention provides GLP-1 fragments which are used in the treatment of glucose metabolism pathologies such as type 2 diabetes, as well as in the treatment of other diseases that are known to benefit from GLP-1 receptor activation.

### SUMMARY OF THE INVENTION

The invention provides a glucagon-like peptide-1 (GLP-1) fragment or a derivative thereof of the formula R7-R8-R9-R10-R11-R12-R13-R14-R15-R16-R17-R18-R19-R20-R21-R22-R23-R24-R25-R26-R27, wherein the glucagon-like peptide-1 fragment or a derivative thereof is between 9 and 21 amino acids in length, wherein the C terminus of the formula is truncated or not truncated, wherein R7 represents histidine, lysine, arginine, phenylalanine, tyrosine, alanine or a non-natural amino acid, R8 is selected from a member of the group consisting of alanine, glutamine, arginine, tyrosine, glycine, valine, histidine or a non-natural amino acid, R9 is selected from a member of the group consisting of glutamate, alanine, leucine, methionine, glutamine, arginine, tyrosine, histidine, aspartate or a non-natural amino acid, R10 represents glycine alanine or a non-natural amino acid, R11 represents threonine, alanine, glutamine, arginine, tyrosine, histidine or a non-natural amino acid, R12 represents phenylalanine, alanine or a non-natural amino acid, R13 represents threonine, alanine or a non-natural amino acid, R14 represents serine, alanine, glutamine, arginine, tyrosine, histidine or a non-natural amino acid, R15 represents aspartate, alanine or a non-natural amino acid, R16 represents truncated, valine, alanine, glutamine, arginine, leucine, tyrosine or a non-natural amino acid, R17 represents truncated, serine, alanine, glutamine, arginine, tyrosine or a non-natural amino acid, R18 represents truncated, serine, alanine, glutamine, arginine, lysine, tyrosine or a non-natural amino acid, R19 represents truncated, tyrosine, alanine, glutamine, arginine, or a non-natural amino acid, R20 represents truncated, leucine, alanine, glutamine, arginine, methionine, tyrosine or a non-natural amino acid, R21 represents truncated, glutamate or a non-natural amino acid, R22 represents truncated, glycine or a non-natural amino acid, R23 represents truncated, glutamine or a non-natural amino acid, R24 represents truncated, alanine or a non-natural amino acid, R25 represents truncated, alanine or a non-natural amino acid, R26 represents truncated, lysine or a non-natural amino acid, and R27 represents truncated, glutamate or a non-natural amino acid, and wherein when any of R16 to R27 are truncated each R group position C-terminal to the truncated R group is also truncated. In one embodiment of the GLP-1 fragment or the derivative thereof, the non-natural amino acid is D-alanine, homoarginine, alpha-aminoisobutyric acid, diethylglycine, 1-aminocyclopentane-1-carboxylic acid, or 1-aminocyclohexane-1-carboxylic acid.

In another embodiment of the GLP-1 fragment or the derivative thereof, the carboxy-terminal residue in each GLP-1 fragment may be either amidated or non-amidated.

In another embodiment of the GLP-1 fragment or the derivative thereof, the glucagon-like peptide-1 fragment or the derivative thereof includes the amino acid sequence of SEQ ID NO:3.

In another embodiment of the GLP-1 fragment or the derivative thereof, the GLP-1 fragment or the derivative thereof also includes one, two, or three small molecules covalently bonded to the glucagon-like peptide fragment or the derivative thereof. In one aspect of this embodiment, the small molecule is a phenylalanine, 4-benzoylphenylalanine, benzylalanine, alanine-o-pentafluorphenyl, biphenylalanine, or T0632 or derivatives of biphenylalanine or T0632.

In another embodiment of the GLP-1 fragment or the derivative thereof, the small molecule is bound to the carboxy terminus of the glucagon-like peptide fragment or the derivative thereof.

In another embodiment of the GLP-1 fragment or the derivative thereof, the glucagon-like peptide fragment or the derivative thereof contains at least 9 amino acids.

In another embodiment of the GLP-1 fragment or the derivative thereof, the glucagon-like peptide fragment or the derivative thereof contains at most 21 amino acids.

In another embodiment of the GLP-1 fragment or the derivative thereof, the glucagon-like peptide fragment or the derivative thereof has an amino acid sequence of SEQ ID NO:2, 3, or 4.

The invention also provides a method of treating a member of the group consisting of glucose metabolism pathology, obesity, congestive heart failure and Alzheimer's disease in a subject in need thereof by administering to the subject a therapeutic amount of one or more glucagon-like peptide fragments or derivatives thereof of the formula R7-R8-R9-R10-R11-R12-R13-R14-R15-R16-R17-R18-R19-R20-R21-R22-R23-R24-R25-R26-R27, wherein the glucagon-like peptide-1 fragment or a derivative thereof is between 9 and 21 amino acids in length, wherein the C terminus of the formula is truncated or not truncated, wherein R7 represents histidine, lysine, arginine, phenylalanine, tyrosine, alanine or a non-natural amino acid, R8 represents alanine, glutamine, arginine, tyrosine, glycine, valine, histidine or a non-natural amino acid, R9 represents glutamate, alanine, leucine, methionine, glutamine, arginine, tyrosine, histidine, aspartate or a non-natural amino acid, R10 represents glycine alanine or a non-natural amino acid, R11 represents threonine, alanine, glutamine, arginine, tyrosine, histidine or a non-natural amino acid, R12 represents phenylalanine alanine or a non-natural amino acid, R13 represents threonine, alanine or a non-natural amino acid, R14 represents serine, atanine, glutamine, arginine, tyrosine, histidine or a non-natural amino acid, R15 represents aspartate, alanine or a non-natural amino acid, R16 represents truncated, valine, alanine, glutamine, arginine, leucine, tyrosine or a non-natural amino acid, R17 represents truncated, serine, alanine, glutamine, arginine, tyrosine or a non-natural amino acid, R18 represents truncated, serine, alanine, glutamine, arginine, lysine, tyrosine or a non-natural amino acid, R19 represents truncated, tyrosine, alanine, glutamine, arginine, or a non-natural amino acid, R20 represents truncated, leucine, alanine, glutamine, arginine, methionine, tyrosine or a non-natural amino acid, R21 represents truncated, glutamate or a non-natural amino acid, R22 represents truncated, glycine or a non-natural amino acid, R23 represents truncated, glutamine or a non-natural amino acid, R24 represents truncated, alanine or a non-natural amino acid, R25 represents truncated, alanine or a non-natural amino acid, R26 represents truncated, lysine or a non-natural amino acid, and R27 represents truncated, glutamate or a non-natural amino acid, and wherein when any of R16 to R27 are truncated each R group position C-terminal to the truncated R group is also truncated. In one embodiment of the method of treating a glucose metabolism pathology in a subject in need thereof, the glucose metabolism pathology is type 1 diabetes or type 2 diabetes.

In another embodiment of the method of treating a glucose metabolism pathology in a subject in need thereof, the non-natural amino acid is D-alanine, homoarginine, alpha-aminoisobutyric acid, diethylglycine, 1-aminocyclopentane-1-carboxylic acid, or 1-aminocyclohexane-1-carboxylic acid.

In another embodiment of the method of treating a glucose metabolism pathology in a subject in need thereof, the carboxy-terminal residue in each of the one or more GLP-1 fragments or derivatives thereof may be either amidated or non-amidated.

In another embodiment of the method of treating a glucose metabolism pathology in a subject in need thereof, the one or more glucagon-like peptide fragments or derivatives thereof include the amino acid sequence of SEQ ID NO:3.

In another embodiment of the method of treating a glucose metabolism pathology in a subject in need thereof, the one or more glucagon-like peptide fragments or derivatives thereof also include a small molecule covalently bonded to the one or more glucagon-like peptide fragments or the derivatives thereof. In one aspect of this embodiment, the small molecule is phenylalanine, 4-benzoylphenylalanine, benzylalanine, alanine-o-pentafluorphenyl, biphenylalanine, or T0632 or derivatives of biphenylalanine or T0632. In another aspect of this embodiment, the small molecule is bound to the carboxy termini of the one or more glucagon-like peptide fragments or derivatives thereof.

In another embodiment of the method of treating a glucose metabolism pathology in a subject in need thereof, the one or more glucagon-like peptide fragments or derivatives thereof contain at least 9 amino acids.

In another embodiment of the method of treating a glucose metabolism pathology in a subject in need thereof, the one or more glucagon-like peptide fragments or derivatives thereof contains at most 21 amino acids.

In another embodiment of the method of treating a glucose metabolism pathology in a subject in need thereof, the one or more glucagon-like peptide fragments or derivatives thereof are selected from SEQ ID NO:2, 3, or 4.

In another embodiment of the method of treating a glucose metabolism pathology in a subject in need thereof, the method further includes the step of administering one or more of the following: a lipid modulating drug, an antidiabetic agent, an antidepressant, an appetite suppressant, and an anti-obesity agent. In one aspect of this embodiment, the lipid modulating agent is an hypolipidemic agent, an HMG-CoA reductase inhibitor, fibrate, an MTP inhibitor, or a squalene synthetase inhibitor. In another aspect of this embodiment, the antidiabetic agent is selected from biguanides, sulfonyl ureas, glucosidase inhibitors, thiazolidinediones, aP2 inhibitors, PPAR agonists, SGLT2 inhibitors, insulin, inhibitors of either dipeptidyl peptidase IV (DPP IV) or neutral aminopeptidase 24.11, or meglitinide. In another aspect of this embodiment, the antidepressant is fluoxetine or desipramine. In another aspect of this embodiment, the appetite suppressant is sibutramine. In another aspect of this embodiment, the anti-obesity agent is orlistat or a β3 agonist.

In another embodiment of the method of treating a glucose metabolism pathology, at least one symptom of type 1 or type 2 diabetes is treated or reduced as a result of the administration of a therapeutic amount of one or more glucagon-like peptide fragments or derivatives thereof. In one aspect of this embodiment, the symptom is frequent urination, excessive thirst, extreme hunger, unusual weight loss, increased fatigue, irritability, blurry vision, genital itching, odd aches and pains, dry mouth, dry or itchy skin, impotence, vaginal yeast infections, poor healing of cuts and scrapes, excessive or unusual infections, hyperglycemia, loss of glycemic control, fluctuations in postprandial blood glucose, fluctuations in blood glucagon, or fluctuations in blood triglycerides.

The invention also provides an antibody which selectively binds to a glucagon-like peptide fragment or a derivative thereof with an amino acid sequence of SEQ ID NOs:2, 3, or 4. In one embodiment, the antibody is a monoclonal antibody. In another embodiment, the antibody is a polyclonal antibody.

The invention also provides a kit for treating a patient having a glucose metabolism pathology, including a therapeutically effective dose of a glucagon-like peptide fragment or a derivative thereof and at least one agent selected from a lipid modulating drug, an antidiabetic agent, an antidepressant, an appetite suppressant, and an anti-obesity agent, either in the same or separate packaging, and instructions for its use.

### BRIEF DESCRIPTION OF THE DRAWINGS

FIG. 1A is a line graph showing the stimulation of the wild-type GLP-1 receptor leading to cAMP production in COS-7 cells in the presence of a 14 amino acid, carboxy-terminally amidated fragment of GLP-1 (closed circles) and in the presence of the same 14 amino acid fragment with the carboxy-terminal tethering of a biphenylalanine (open circles).

FIG. 1B is a line graph showing the stimulation of a hyperactive GLP-1 receptor leading to cAMP production in COS-7 cells in the presence of a 14 amino acid, carboxy-terminally amidated fragment of GLP-1 (closed squares) and in the presence of the same 14 amino acid fragment with the carboxy-terminal tethering of a biphenylalanine (open squares).

### DETAILED DESCRIPTION OF THE INVENTION

The invention provides GLP-1 fragments from about 9 to about 21 amino acids in length. These fragments comprise an amino acid sequence of from about 9 to about 21 amino acids from about amino acid 7 to about amino acid 28 of GLP-1. In some embodiments, the fragments are from about 9 to about 14 amino acids in length. In a preferred embodiment of the GLP-1 fragment of the invention, the fragment comprises residues 7-20 of the GLP-1 peptide. This embodiment may alternately be referred to as either GLP-1 (1-14) or GLP-1 (7-20). In both instances, the GLP-1 fragment referred to is that of SEQ ID NO: 3. These fragments may be synthetically derivatized at any one or more of their amino acid residues. These fragments may also be derivatized through the substitution of amino acids at any position on the fragments. The GLP-1 (7-20) fragment may be synthetically derivatized at any of its amino acid residues, but preferably it is synthetically derivatized at its carboxy terminus. Optionally it is synthetically derivatized with a small molecule such as phenylalanine, 4-benzoylphenylalanine, benzylalanine, alanine-o-pentafluorphenyl, biphenylalanine, or T0632 or derivatives of biphenylalanine or T0632.

The invention also provides a method of preventing or treating diabetes with GLP-1 fragments or derivatives thereof. Potential advantages of using GLP-1 fragments over full length, (7-36) or (7-37) GLP-1 include increased stability of the fragments of the invention in vivo and their ability to be administered orally. The GLP-1 peptides of the invention may be administered to treat type 1 (insulin dependent) or type 2 (non-insulin dependent) diabetes mellitus.

The invention also provides combination compositions and therapies for the treatment of diabetes. The combination compositions include GLP-1 fragments and another composition which affects glucose metabolism.

The invention also provides kits for use in these methods.

The invention also provides antibodies for the determination of a therapeutic level of GLP-1 fragments and derivatives thereof in vivo.

### GLP-1 Fragments and Derivatives Thereof

Glucagon-like peptide-1 is an endogenous hormone that plays a role in maintaining blood glucose homeostasis. The sequence of full length GLP-1 (1-37) is shown in Table 1.

TABLE 1
Unprocessed GLP-1 (1-37) amino acid sequence
amino acid sequence.

HDEFERHAEGTFTSDVSSYLEGQAAKEFIAWLVKGRG
(SEQ ID NO: 1)

GLP-1 (1-37) is full-length, unprocessed, biologically inactive GLP-1. The amino-terminally truncated, biologically active forms, on which the peptide fragments of the invention are based, start with the histidine residue in position 7: GLP-1 (7-36) amide or GLP-1 (7-37).

A subset of GLP-1 fragments of the invention include the fragments shown in Table 2.

TABLE 2
Exemplary GLP-1 fragments.

| | Number scheme from unprocessed GLP-1 | | | | | | | | | | | | | | | | | | | | |
|---|---|---|---|---|---|---|---|---|---|---|---|---|---|---|---|---|---|---|---|---|---|
| | 7 | 8 | 9 | 10 | 11 | 12 | 13 | 14 | 15 | 16 | 17 | 18 | 19 | 20 | 21 | 22 | 23 | 24 | 25 | 26 | 27 |
| 7-27 peptide (SEQ ID | H | A | E | G | T | F | T | S | D | V | S | S | Y | L | E | G | Q | A | A | K | E |
| NO: 2) | | | | | | | | | | | | | | | | | | | | | |
| 7-20 peptide (SEQ ID | H | A | E | G | T | F | T | S | D | V | S | S | Y | L | | | | | | | |
| NO: 3) | | | | | | | | | | | | | | | | | | | | | |
| 7-15 peptide (SEQ ID | H | A | E | G | T | F | T | S | D | | | | | | | | | | | | |
| NO: 4) | | | | | | | | | | | | | | | | | | | | | |
| Potential C-Terminal | | | | | | | | | | X | X | X | X | X | X | X | X | X | X | X | X |
| Deletions | | | | | | | | | | | | | | | | | | | | | |
| Substitutions with | A | | A | A | A | A | A | A | A | A | A | A | A | A | | | | | | | |
| natural amino acids | K | Q | Q | | Q | | | Q | | Q | Q | Q | Q | Q | | | | | | | |
| (single or in | R | R | R | | R | | | R | | R | R | R | R | R | | | | | | | |
| combination) | F | Y | Y | | Y | | | Y | | Y | Y | Y | | Y | | | | | | | |
| | Y | H | H | | H | | | H | | L | | K | | M | | | | | | | |
| | | V | D | | | | | | | | | | | | | | | | | | |
| | | G | L | | | | | | | | | | | | | | | | | | |
| | | | M | | | | | | | | | | | | | | | | | | |
| Substitutions with | Da | Da | Da | Da | Da | Da | Da | Da | Da | Da | Da | Da | Da | Da | Da | Da | Da | Da | Da | Da | Da |
| non-natural amino | Hr | Hr | Hr | Hr | Hr | Hr | Hr | Hr | Hr | Hr | Hr | Hr | Hr | Hr | Hr | Hr | Hr | Hr | Hr | Hr | Hr |
| acids (single or in | Ab | Ab | Ab | Ab | Ab | Ab | Ab | Ab | Ab | Ab | Ab | Ab | Ab | Ab | Ab | Ab | Ab | Ab | Ab | Ab | Ab |
| combination) | Dg | Dg | Dg | Dg | Dg | Dg | Dg | Dg | Dg | Dg | Dg | Dg | Dg | Dg | Dg | Dg | Dg | Dg | Dg | Dg | Dg |
| | A5 | A5 | A5 | A5 | A5 | A5 | A5 | A5 | A5 | A5 | A5 | A5 | A5 | A5 | A5 | A5 | A5 | A5 | A5 | A5 | A5 |
| | A6 | A6 | A6 | A6 | A6 | A6 | A6 | A6 | A6 | A6 | A6 | A6 | A6 | A6 | A6 | A6 | A6 | A6 | A6 | A6 | A6 |

Da = D-alanine, Hr = homoarginine, Ab = alpha-aminoisobutyric acid, Dg = diethylglycine, A5 = 1-aminocyclopentane-1-carboxylic acid, A6 = 1-aminocyclohexane-1-carboxylic acid.

As shown in Table 2, the fragments of the invention contemplate fragments as long as GLP-1 (7-27) and as short as GLP-1 (7-15). Fragments may end with any C-terminal GLP-1 residue from 15-27. Further residues 7-20 of GLP-1 may be substituted with other amino acids as shown in Table 2. It is crucial that amino acid 7 not be methionine. Histidine is the preferred residue at position 7, but other aromatic/positively charged residues (lysine, arginine, phenylalanine, tyrosine) are also possible, as well as alanine. The most carboxy-terminal residue in each peptide may or may not be amidated.

GLP-1 fragments specifically bind with the GLP-1 receptor and increase its activity. Through this and other mechanisms, GLP-1 fragments are used to treat and prevent diabetes and other pathologies related to glucose, carbohydrate and fat metabolism.

In a specific embodiment, GLP-1 fragments are derivatized. For example, the underivatized GLP-1 (7-20) fragment specifically binds with the GLP-1 receptor (see Example 1, below) but with lower affinity than full length GLP-1. In one embodiment, derivatives of GLP-1 (7-20) or any of the GLP-1 fragments bind with similar or greater affinity to the GLP-1 receptor as full length GLP-1. Also, the derivatives are functionally active, i.e., capable of exhibiting one or more functional activities associated with GLP-1. Derivatives of GLP-1 fragments can be tested for the desired activity by procedures known in the art, including but not limited to, using appropriate cell lines, isolated pancreatic islets, animal models, and clinical trials.

In particular, GLP-1 fragment derivatives can be made via altering GLP-1 fragment sequences by substitutions, insertions or deletions that provide for functionally improved molecules. Due to the degeneracy of nucleotide coding sequences, other DNA sequences which encode substantially the same amino acid sequence as a GLP-1 fragment or a derivative thereof may be used in the practice of the present invention. These include, but are not limited to, nucleic acid sequences comprising all or portions of GLP-1 fragments or derivatives thereof that are altered by the substitution of different codons that encode a functionally equivalent amino acid residue within the sequence, thus producing a silent change. Likewise, the GLP-1 fragments and derivatives thereof of the invention include, but are not limited to, those containing, as a primary amino acid sequence, all or part of the amino acid sequence of residues 7-27 of full length GLP-1 including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. GLP-1 fragment derivatives of the invention also include, but are not limited to, those containing, as a primary amino acid sequence, all or part of the amino acid sequence of GLP-1 (7-27) including altered sequences in which amino acid residues are substituted for residues with similar chemical properties. In a specific embodiment, 1, 2, 3, 4, or 5 amino acids are substituted.

Derivatives of GLP-1 fragments include, but are not limited to, those proteins which are substantially homologous to GLP-1 (7-27) or fragments thereof, or whose encoding nucleic acid is capable of hybridizing to the GLP-1 (7-27) nucleic acid sequence.

In a specific embodiment, chimeric or fusion proteins may be used in the method of the invention. As used herein, a “chimeric protein” or “fusion protein” comprises GLP-1 fragments or derivatives thereof operatively-linked to a non-GLP-1 fragment polypeptide. Within such a fusion protein, the GLP-1 fragments or derivatives thereof can correspond to all or a portion of GLP-1 fragments or derivatives thereof. In one embodiment, a GLP-1 fragment fusion protein comprises at least one biologically-active portion of a GLP-1 fragment. Within the fusion protein, the GLP-1 fragment and the non-GLP-1 fragment are “operatively-linked”, that is they are fused in-frame with one another. The non-GLP-1 fragment can be fused to the N-terminus or C-terminus of the GLP-1 fragment. For example, the fusion protein may be a GLP-1 fragment containing a heterologous signal sequence at its N-terminus. In certain host cells (e.g., mammalian host cells), expression and/or secretion of GLP-1 fragments can be increased through use of a heterologous signal sequence. In yet another example, the fusion protein is a GLP-1 fragment-immunoglobulin fusion protein in which the GLP-1 fragment sequences are fused to sequences derived from a member of the immunoglobulin protein family. The GLP-1 fragment-immunoglobulin fusion proteins can be incorporated into pharmaceutical compositions and administered to a subject to inhibit an immunological response according to the present invention.

A GLP-1 fragment derivative, or GLP-1 fragment chimeric or fusion protein for use in the method of the invention may be chemically modified for the purpose of improving bioavailability, and increasing efficacy, solubility and stability. For example, the fragment may be covalently or non-covalently linked to polyethylene glycol (PEG).

A GLP-1 fragment derivative, or GLP-1 fragment chimeric or fusion protein for use in the method of the invention can be produced by standard recombinant DNA techniques. For example, DNA fragments coding for the different polypeptide sequences may be ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. Furthermore, the gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence [see, e.g., Ausubel, et al. (eds.) CURRENT PROTOCOLS IN MOLECULAR BIOLOGY, John Wiley & Sons, (1992)]. Moreover, many expression vectors are commercially available that already encode a fusion moiety (e.g., a GST polypeptide). A GLP-1 fragment or a derivative thereof-encoding nucleic acid can be cloned into such an expression vector such that the fusion moiety is linked in-frame to the GLP-1 fragment or the derivative thereof. The fusion protein can be a GLP-1 fragment or a derivative thereof fused to a His tag or epitope tag (e.g. V5) to aid in the purification and detection of the recombinant GLP-1 fragment or a derivative thereof, or to mask the immune response in a subject.

In some embodiments, a GLP-1 fragment or a derivative thereof can be modified so that it has an extended half-life in vivo using any methods known in the art. For example, Fc fragment of human IgG, a small molecule or inert polymer molecules such as high molecular weight polyethyleneglycol (PEG) can be attached to GLP-1 fragments or derivatives thereof with or without a multifunctional linker either through site-specific conjugation of the PEG to the N- or C-terminus of the protein or via epsilon-amino groups present on lysine residues. Linear or branched polymer derivatization that results in minimal loss of biological activity can be used. The degree of conjugation can be closely monitored by SDS-PAGE and mass spectrometry to ensure proper conjugation of PEG molecules to GLP-1 fragment or a derivative thereof. Unreacted PEG can be separated from GLP-1-PEG conjugates by size-exclusion or by ion-exchange chromatography. PEG-derivatized conjugates can be tested for in vivo efficacy using methods known to those of skill in the art.

The GLP-1 fragments or derivatives thereof can be derivatized with one or more small molecules at any residue of the fragment or the derivative thereof. Preferably these small molecules are attached to the carboxy-terminus of the GLP-1 fragments or derivatives thereof. Small molecules for derivatization include but are not limited to biphenylalanine, phenylalanine, 4-benzoylphenylalanine, benzylalanine, alanine-o-pentafluorphenyl, or biphenylalanine derivatives.

Furthermore, small molecules for derivatization of GLP-1 fragments or derivatives thereof include compound T0632 or its analogues. This class of compounds that may be formulated as pharmaceutical compositions have the formula:

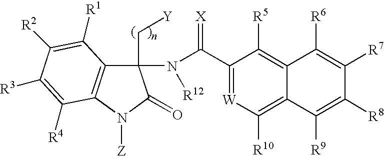

or a pharmaceutically acceptable salt thereof, such that W is N or CR¹¹, X is O or S; Y is CO₂H or SO₃H; Z is a substituted or unsubstituted C₆ aryl; n is 1-3; each of R¹-R¹⁰ is, independently, a H, C₁-C₆ alkyl, halogen, NO₂, OH, C₁₋₆ alkoxy, CO₂H, CO₂R¹³, NR¹⁴R¹⁵, CN, or CF₃; and each of R¹¹ and R¹² is a H or C₁-C₆ alkyl. Each of R¹³, R¹⁴, and R may be a H, C₁₋₆ alkyl, or C₁₋₄ alkaryl. These above-defined molecules may also be used to derivatize any amino acid of a GLP-1 fragment or a derivative thereof. Preferably, these molecules are used to derivatize the C-terminus of a GLP-1 fragment or a derivative thereof.

The GLP-1 fragment or a derivative thereof of the invention may also be derivatized with a compound of the formula:

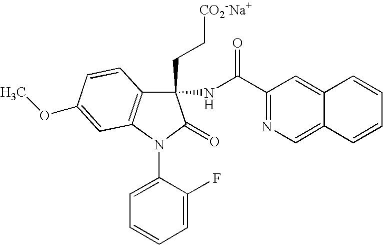

This compound is also referred to as T0632 (sodium (S)-3-[1-(2-fluorophenyl)-2,3-dihydro-3-[(3-isoquinolinyl)-carbonyl]amino-6-methoxy-2-oxo-1-H-indole)propanoate). Preferably, this small molecule is attached to the carboxy-terminus of a GLP-1 peptide fragment or derivative thereof.

Another small molecule which is used to derivatize GLP-1 fragments and derivatives thereof is shown below:

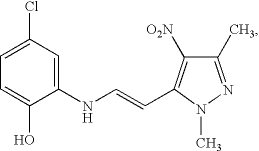

This or any other compound found in U.S. Pat. No. 5,807,883, (hereby incorporated by reference, in its entirety) may be used to derivatize GLP-1 fragments and derivatives thereof of the invention.

### Methods of the Invention and Agents Useful Therein

Overview of the Methods of the Invention

The present invention provides GLP-1 fragment based, GLP-1 fragment derivative based and combination based therapies and methods for treating type 1 or type 2 diabetes mellitus and related conditions in which there is a lack of or diminished insulin production in a patient.

In one embodiment, the combination based therapies of the invention include GLP-1 fragments or derivatives thereof which are administered in combination with one or more members of the group of lipid modulating drugs, antidiabetic agents, antidepressants, appetite suppressants, and/or anti-obesity agents. Lipid modulating drugs include such compositions as HMG-CoA reductase inhibitors, fibrate, MTP inhibitors, squalene synthetase inhibitors and other hypolipidemic agents.

Antidepressants include such compositions as fluoxetine and desipramine.

Appetite suppressants include compositions such as sibutramine. Optionally they are used in combination with anti-obesity agents such as orlistat, or a β3 agonist.

Hypolipidemic agents include thiazolidinediones, MTP inhibitors, HMG CoA reductase inhibitors, squalene synthetase inhibitors, fibric acid derivatives, ACAT inhibitors, cholesterol absorption inhibitors, ileal Na⁺/bile acid cotransporter inhibitors, bile acid sequestrants, and/or nicotinic acid and derivatives thereof.

MTP inhibitors employed herein include MTP inhibitors disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. Pat. No. 5,962,440 incorporated herein, by reference. Most preferred MTP inhibitors to be employed in accordance with the present invention include preferred MTP inhibitors as set out in U.S. Pat. Nos. 5,739,135 and 5,712,279, and U.S. Pat. No. 5,760,246.

The most preferred MTP inhibitor is

9-[4-[4-[[2-(2,2,2-Trifluoroethoxy)benzoyl]amino]-1-piperidinyl]butyl]-N-(2,2,2-trifluoroethyl)-9H-fluorene-9-carboxamide.

For oral administration, a satisfactory result may be obtained employing the MTP inhibitor in an amount within the range of from about 0.01 mg/kg to about 100 mg/kg and preferably from about 0.1 mg/kg to about 75 mg/kg, one to four times daily.

A preferred oral dosage form, such as tablets or capsules, will contain the MTP inhibitor in an amount of from about 1 to about 500 mg, preferably from about 2 to about 400 mg, and more preferably from about 5 to about 250 mg, one to four times daily.

For parenteral administration, the MTP inhibitor will be employed in an amount within the range of from about 0.005 mg/kg to about 10 mg/kg and preferably from about 0.005 mg/kg to about 8 mg/kg, one to four times daily.

Hypolipidemic agents include HMG CoA reductase inhibitors which include, but are not limited to, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, and simvastatin and related compounds as disclosed in U.S. Pat. Nos. 4,448,784 and 4,450,171. Other HMG CoA reductase inhibitors which may be employed herein include, but are not limited to, fluvastatin, disclosed in U.S. Pat. No. 5,354,772, cerivastatin disclosed in U.S. Pat. Nos. 5,006,530 and 5,177,080, atorvastatin disclosed in U.S. Pat. Nos. 4,681,893, 5,273,995, 5,385,929 and 5,686,104, pyrazole analogs of mevalonolactone derivatives as disclosed in U.S. Pat. No. 4,613,610, indene analogs of mevalonolactone derivatives as disclosed in PCT application WO 86/03488, 6-[2-(substituted-pyrrol-1-yl)-alkyl)pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054, 3-carboxy-2-hydroxy-propane-phosphonic acid derivatives as disclosed in French Patent No. 2,596,393, 2,3-disubstituted pyrrole, furan and thiophene derivatives as disclosed in European Patent Application No. 0221025, naphthyl analogs of mevalonolactone as disclosed in U.S. Pat. No. 4,686,237, octahydronaphthalenes such as disclosed in U.S. Pat. No. 4,499,289, keto analogs of mevinolin (lovastatin) as disclosed in European Patent Application No. 0,142,146 A2, as well as other known HMG CoA reductase inhibitors. In addition, phosphinic acid compounds useful in inhibiting HMG CoA reductase suitable for use herein are disclosed in GB 2205837.

For oral administration, a satisfactory result may be obtained employing an HMG CoA reductase inhibitor, for example, pravastatin, lovastatin, simvastatin, atorvastatin, fluvastatin or cerivastatin in dosages employed as indicated in the Physician's Desk Reference, such as in an amount within the range of from about 1 to 2000 mg, and preferably from about 4 to about 200 mg. The squalene synthetase inhibitor may be employed in dosages in an amount within the range of from about 10 mg to about 2000 mg and preferably from about 25 mg to about 200 mg.

A preferred oral dosage form, such as tablets or capsules, will contain the HMG CoA reductase inhibitor in an amount from about 0.1 to about 100 mg, preferably from about 5 to about 80 mg, and more preferably from about 10 to about 40 mg.

Other hypolipidemic agents suitable for use herein include, but are not limited to, fibric acid derivatives, such as fenofibrate, gemfibrozil, clofibrate, bezafibrate, ciprofibrate, clinofibrate and the like, probucol, and related compounds as disclosed in U.S. Pat. No. 3,674,836, probucol and gemfibrozil being preferred, bile acid sequestrants such as cholestyramine, colestipol and DEAE-Sephadex (SECHOLEX™, POLICEXIDE™), as well as lipostabil (Rhone-Poulenc), Eisai E-5050 (an N-substituted ethanolamine derivative), imanixil (HOE-402), tetrahydrolipstatin (THL), istigmastanylphos-phorylcboline (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives such as disclosed in U.S. Pat. No. 4,759,923, quaternary amine poly(diallyldimethylammonium chloride) and ionenes such as disclosed in U.S. Pat. No. 4,027,009, and other known serum cholesterol lowering agents.

Another hypolipidemic agent may be an ACAT inhibitor such as disclosed in, Drugs of the Future 24, 9-15 (1999), (Avasimibe); “The ACAT inhibitor, Cl-1011 is effective in the prevention and regression of aortic fatty streak area in hamsters”, Nicolosi et al, Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al, Bioorg. Med. Chem. Lett. (1996), 6(1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62. The hypolipidemic agent may also be a cholesterol absorption inhibitor preferably Schering-Plough's SCH48461 as well as those disclosed in Atherosclerosis 115, 45-63 (1995) and J. Med. Chem. 41, 973 (1998). The hypolipidermic agent may also be an ileal Na⁺/bile acid cotransporter inhibitor such as disclosed in Drugs of the Future, 24, 425-430 (1999). Preferred hypolipidemic agents include pravastatin, lovastatin, simvastatin, atorvastatin, fluvastatin and cerivastatin.

The squalene synthetase inhibitors suitable for use herein include, but are not limited to, α-phosphonosulfonates disclosed in U.S. Pat. No. 5,712,396, those disclosed by Biller et al, J. Med. Chem., 1988, Vol. 31, No. 10, pp 1869-1871, including isoprenoid (phosphinylmethyl)phosphonates as well as other squalene synthetase inhibitors as disclosed in U.S. Pat. Nos. 4,871,721 and 4,924,024 and in Biller, S. A., Neuenschwander, K., Ponpipom, M. M., and Poulter, C. D., Current Pharmaceutical Design, 2, 1-40 (1996). In addition, other squalene synthetase inhibitors suitable for use herein include the terpenoid pyrophosphates disclosed by P. Ortiz de Montellano et al, J. Med. Chem., 1977, 20, 243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J.A.C.S., 1987, 109, 5544 and cyclopropanes reported by Capson, T. L., PhD dissertation, June, 1987, Dept. Med. Chem. U of Utah, Abstract, Table of Contents, pp 16, 17, 40-43, 48-51, Summary.

A preferred oral dosage form, such as tablets or capsules will contain the squalene synthetase inhibitor in an amount of from about 10 to about 500 mg, preferably from about 25 to about 200 mg.

Antidiabetic agents include an oral antihyperglycemic agent preferably a biguanide such as metformin or phenformin or salts thereof. The antidiabetic agent may also preferably be a sulfonylurea such as glyburide (also known as glibenclamide), glimepiride (disclosed in U.S. Pat. No. 4,379,785), glipizide, gliclazide or chlorpropamide, other known sulfonylureas or other antihyperglycemic agents which act on the ATP-dependent channel of the .beta.-cells, with glyburide and glipizide being preferred. The oral antidiabetic agent may also be a glucosidase inhibitor such as acarbose (disclosed in U.S. Pat. No. 4,904,769) or miglitol (disclosed in U.S. Pat. No. 4,639,436), which may be administered in a separate oral dosage form.

Antidiabetic agents also include insulin, inhibitors of dipeptidyl peptidase IV (DPP IV) and inhibitors of neutral aminopeptidase 24.11 (Plamboek et al., Diabetologia 2005, Jul. 16 [Epub ahead of print]) and may also be employed in combination with the GLP-1 fragments of the invention or derivatives thereof. Insulin may be administered from about 10 to about 100 units/day. Primarily, insulin is administered intravenously. DPP IV inhibitors (Baggio L L and Drucker D J, Treat Endocrinol. 2002, 1:117-25; McIntosh C H et al., Regul Pept. 2005, 128:159-65; Nielsen L L, Drug Discov Today. 2005, 10: 703-10) including but not limited to NVP-PP728, LAF237 (Vildagliptin), or MK-0431 may be administered at doses between about 5 and 500 mg/day to delay enzymatic degradation of GLP-1 and related peptides. The preferred route of application of DPP IV inhibitors is orally, between one and four times/day. Inhibitors of neutral aminopeptidase 24.11 such as candoxatril delay the caroxy-terminal degradation of GLP-1 peptides. These compounds may be combined with GLP-1 fragments or derivatives thereof, either alone or together with DPPIV inhibitors. Inhibitors of neutral aminopeptidase 24.11 may be applied either orally or intravenously, at doses between about 5 and 500 mg/day.

The GIP-1 fragments or derivatives thereof may be employed in combination with a thiazolidinedione oral anti-diabetic agent or other insulin sensitizers (which has an insulin sensitivity effect in NIDDM patients) such as troglitazone (Wamer-Lambert's Rezulin®, disclosed in U.S. Pat. No. 4,572,912), rosiglitazone (SKB), pioglitazone (Takeda), Mitsubishi's MCC-555 (disclosed in U.S. Pat. No. 5,594,016), Glaxo-Welcome's GI-262570, englitazone (CP-68722, Pfizer), or darglitazone (CP-86325, Pfizer). The sulfonylurea and thiazolidinedione in amounts of less than about 150 mg oral antidiabetic agent may be incorporated in a single tablet with the described GLP-1 fragments or derivatives thereof.

Where present, metformin, the sulfonylureas, such as glyburide, glimepiride, glipyride, glipizide, chlorpropamide and gliclazide and the glucosidase inhibitors acarbose or miglitol or insulin may be employed in formulations as described above and in amounts and dosing as indicated in the Physician's Desk Reference.

Where present, metformin or salts thereof may be employed in amounts within the range from about 500 to about 2000 mg per day which may be administered in single or divided doses one to four times daily.

Where present, the thiazolidinedione anti-diabetic agent may be employed in amounts within the range from about 0.01 to about 2000 mg/day which may be administered in single or divided doses one to four times per day.

Where present insulin may be employed in formulations, amounts and dosing as indicated by the Physician's Desk Reference.

The antidiabetic agent may also be a PPARaly dual agonist such as disclosed by Murakami et al, “A Novel Insulin Sensitizer Acts As a Coligand for Peroxisome Proliferation—Activated Receptor Alpha (PPARα) and PPARγ. Effect on PPARα Activation on Abnormal Lipid Metabolism in Liver of Zucker Fatty Rats”, Diabetes 47, 1841-1847 (1998).

The antidiabetic agent may be an aP2 inhibitor such as disclosed in U.S. application Ser. No. 09/391,053, filed Sep. 7, 1999, and U.S. provisional application No. 60/127,745, filed Apr. 5, 1999 (attorney file LA27*), employing dosages as set out herein.

The antidiabetic agent may be an SGLT2 inhibitor such as disclosed in U.S. provisional application 60/158,773 filed Oct. 12, 1999 (Attorney file LA0049*).

The dose administered must be carefully adjusted according to age, weight and condition of the patient, as well as the route of administration, dosage form and regimen and the desired result.

The above-mentioned U.S. patents, publications and applications are incorporated herein by reference. The amounts and dosages employed will be as indicated in the Physician's Desk Reference and/or in the patents set out above.

The dosages and formulations for the hypolipidemic agents, lipid modulating drugs, antidiabetic agents, antidepressants, appetite suppressants and anti-obesity agents to be employed, where applicable, will be as set out in the latest edition of the Physicians' Desk Reference.

The compositions described above may be administered in the dosage forms as described above in single or divided doses of one to four times daily. It may be advisable to start a patient on a low dose combination and work up gradually to a high dose combination.

The present invention also provides single agent therapies for treating diabetes and related conditions. These single agent therapies include methods for administering GLP-1 fragments or derivatives thereof that treat diabetes and other diseases and conditions relating to aberrant glucose regulation. Diseases and conditions amenable to treatment with this methodology, include but are not limited to type 1 diabetes and type 2 diabetes.

There is a demonstrated need for the therapeutic benefits provided by the present invention. There are new insulin formulations and evidence to support that intensive insulin therapy prevents deaths and reduces the rate of blindness, amputations, and kidney failure necessitating dialysis. However, intensive insulin therapy utilizing modern modalities of multiple insulin injections and continuous insulin delivery via pump therapy is associated with a two-to-three fold increased risk of serious hypoglycemia requiring assistance from another person. In a clinical study setting, despite normalization of glucose in type 1 diabetes patients by means of intravenous insulin and glucose, the standard deviation in glucose levels, both high and low, is significantly wider than non-diabetic study subjects with the same average glucose over a 24-hour period.

Thus, despite insulin's availability and new technologies, including new formulations of human insulin, self blood glucose monitoring systems, continuous glucose sensors and pump therapy, normal glucose control is not approximated by current therapies. Moreover, the underlying mechanisms causing diabetes are not impacted by the current therapies available for patients with type 1 or type 2 diabetes and conditions in which there is no or diminished insulin production.

The combination therapies and related methods of the invention involve the administration of GLP-1 fragments or derivatives thereof or co-administration of GLP-1 fragments or derivatives thereof with one or more members of the group of lipid modulating drugs, antidiabetic agents, antidepressants, appetite suppressants, and/or anti-obesity agents. Lipid modulating drugs include such compositions as HMG-CoA reductase inhibitors, fibrate, MTP inhibitors, squalene synthetase inhibitors and other hypolipidemic agents. As used herein, an agent is “co-administered” or “used in combination” with another agent (also referred to herein as, “agent”) when the two or three agents are administered as part of the same course of therapy. In one embodiment, a first agent is first administered prior to administration of the second agent, and treatment with both is continued throughout the course of therapy. In another embodiment, the second agent is administered after the initiation or completion of the therapy involving the first agent. In other embodiments, the first agent is administered contemporaneously with the initiation of the therapy with the second agent. In another embodiment, a third agent is administered contemporaneously or before or after the administration of the first or second agent or both.

Diseases and Conditions Amenable to Treatment

The GLP-1 fragment or GLP-1 fragment derivative therapies or combination therapies of the present invention can be used to treat any mammal, including humans and animals, suffering from a disease, symptom, or condition related to a pathology related to glucose or carbohydrate metabolism. Such diseases and conditions include type 2 diabetes, type 1 diabetes mellitus, pre-diabetes, including but not limited to pre-diabetes in a type 1 patient as manifested by antibodies (anti-GAD65n and others) specific for type 1 diabetes, and latent autoimmune diabetes of adulthood. Moreover, the present invention can be practiced with therapeutic benefit for patients newly diagnosed as having type 1 diabetes, the siblings and first degree relatives of patients with type 1 diabetes, and people with positive antibodies and other autoimmune conditions that indicate a predilection to type 1 diabetes. In one embodiment the methods of the invention are practiced to reverse diabetes in a patient in need of such treatment.

The combination therapies and related methods and compositions of the invention can also be employed as adjunctive therapy to insulin therapy in type 1 or type 2 diabetes in children and adults, to ameliorate glucose swings among patients with diabetes, and in patients with poorly controlled diabetes, hypoglycemic unawareness, and recurrent hypoglycemia in type 1 or type 2 diabetes.

The GLP-1 fragment or derivative thereof therapies and related methods and compositions of the invention can be used to treat patients having newly diagnosed type 1 diabetes, type 2 diabetes in children and adults with recurrent hypoglycemia, type 1 diabetes being concurrently treated with insulin therapy, and poorly controlled type 1 or type 2 diabetes. In some patients, both children and adults, the methods and compositions of the invention can reverse type 2 diabetes. The methods and compositions of the invention can also be used to treat both children and adults having atypical forms of diabetes and patients having the conditions of postprandial hyperglycemia.

Based on the beneficial effect of GLP-1 receptor activation on obesity (Gutzwiller J P et al., Physiol Behav. 2004, 82:17-9), the GLP-1 fragment or derivative thereof therapies and related methods and compositions of the invention can also be used to treat patients who are children as well as adult patients in need of weight loss, including but not limited to achieve weight loss or treat obesity in patients having type 2 diabetes as well as those who do not have type 1 or 2 diabetes. In one embodiment, the methods and compositions of the invention are used to treat a patient having morbid obesity. In other embodiments, the methods and compositions of the invention are used to treat a patient having morbid obesity or patients having anorexia, bulimia, or other eating disorders.

The single agent therapies and related methods and compositions of the invention can also be used to treat children and adults having dysmetabolic syndrome or metabolic syndrome, as well as patients exhibiting the conditions of hypertriglyceridemia with and without diabetes and postprandial hypertriglyceridemia. In one embodiment, these methods are practiced to treat polycystic ovarian syndrome in a patient in need of such treatment.

Other patients that can benefit from the GLP-1 fragment or derivative thereof therapies and related methods of the invention include children and adult patients diagnosed as having conditions such as fasting hyperglycemia, impaired fasting glucose, impaired glucose tolerance, and hypoglycemic conditions generally.

The GLP-1 fragment or derivative thereof therapies and related methods and compositions of the invention can also be used to treat patients having recurrent pancreatitis or pancreatic cancer and can be used in all modalities of auto islet regeneration.

Furthermore, GLP-1 fragment or GLP-1 fragment derivative therapies or combination therapies of the present invention can be used in the treatment of other diseases that are known to benefit from therapy with GLP-1 receptor agonists, including but not limited to congestive/ischemic heart failure (Nikolaidis L A et al., Circulation 2004, 110:955-961; Bose A K et al., Diabetes 2005, 54: 146-151) and neurodegenerative disorders such as Alzheimer's disease (Perry T and Greig N H, J Alzheimers Dis. 2002, 4:487-96).

### Synergy/Additivity

Synergy is defined as the interaction of two or more agents so that their combined effect is greater than the sum of their individual effects. For example, if the effect of drug A alone in treating a disease is 25%, and the effect of drug B alone in treating a disease is 25%, but when the two drugs are combined the effect in treating the disease is 75%, the effect of A and B is synergistic.

Additivity is defined as the interaction of two or more agents so that their combined effect is greater than the average of their individual effects. For example, if the effect of drug A alone in treating a disease is 25%, and the effect of drug B alone in treating a disease is 25%, but when the two drugs are combined the effect in treating the disease is greater than 25%, the effect of A and B is additive.

An improvement in the drug therapeutic regimen can be described as the interaction of two or more agents so that their combined effect reduces the incidence of adverse event (AF) of either or both agents used in co-therapy. This reduction in the incidence of adverse effects can be a result of, e.g., administration of lower dosages of either or both agent used in the co-therapy. For example, if the effect of Drug A alone is 25% and has an adverse event incidence of 45% at labeled dose; and the effect of Drug B alone is 25% and has an adverse event incidence of 30% at labeled dose, but when the two drugs are combined at lower than labeled doses of each, if the overall effect is 35%, and the adverse incidence rate is 20%, there is an improvement in the drug therapeutic regimen.

The combination therapies described above have both synergistic and additive effects in the treatment and prevention of various pathologies related to diabetes and carbohydrate and fat metabolism.

### Pharmaceutical Compositions, Dosing and Administration

Dosing and administration of the agents useful in the methods of the invention as described herein provide the lowest toxicity for the agents that delay or prevent the destruction of pancreatic islet cells. Pharmaceutical compositions of the invention provide for kinetic delivery of these agents, ease of delivery, and enhanced efficacy.

The agents useful in the methods of the invention can be administered by a variety of routes. Known agents useful in the methods of the invention can be administered by routes and using pharmaceutical formulations previously developed for other indications. Such delivery routes include, at least for most known agents, oral delivery, topical delivery, including micelle and nanosphere topical delivery systems, subcutaneous delivery including pump-assisted continuous infusion and disposable micro-pumps and micro-needles (including but not limited to those available from Animas Corp.), and buccal delivery.

Of course, the particular route of administration and pharmaceutical formulation of an agent used in the practice of the methods of the invention will be selected by the practitioner based on a patient's disease or condition being treated and the agent employed. A wide variety of pharmaceutical compositions can be employed in the methods of the invention. In some embodiments, extended use preparations can be used for ease of administration and increased efficacy. In one embodiment, one or more of the agents employed in the method is formulated as a micelle.

Often, ease of administration is best achieved by oral delivery. While small molecule pharmaceutical agents can often be readily formulated for oral delivery, peptide and protein-based pharmaceutical agents can be more difficult to formulate for oral delivery. However, suitable formulation technology exists, and in one important aspect, the present invention provides pharmaceutical compositions of proteins and peptides formulated for oral delivery. In one embodiment, the pharmaceutical compositions useful in the methods of the invention suitable for oral delivery are formulated generally in accordance with known Technosphere™ technology developed by MannKind Corp., Eligen® Technology developed by Emisphere, and nasal delivery systems developed by Nastech.

Agents that can be formulated for oral delivery and employed in the methods of the invention include GLP-1 fragments or derivatives thereof. Other oral delivery and encapsulation technology suitable for use in making the pharmaceutical compositions of the invention includes the hepatic delivery vesicle (HDV) and pancreatic delivery vesicle (PDV) technology under development by SDG, Inc. and AMDG, Inc. See the reference Davis et al., 2001, J. Diabetes Comp. 15(5): 227-33, incorporated herein by reference, for a description of the technology. HDV technology, as provided by the present invention, can be used to deliver GLP-1 directly to the liver. PDV technology provides liposomes with a conjugated protein or other molecule on its surface that targets an agent, such as a peptide that stimulates islet cell neogenesis, directly to the pancreas.

### Kits

The invention further relates to kits for treating patients having type 1 or type 2 diabetes or other glucose metabolism disorders, comprising a therapeutically effective dose of a GLP-1 fragment or derivative thereof. Optionally, the kit may also contain one or more lipid modulating drugs, antidiabetic agents, antidepressants, appetite suppressants, and anti-obesity agents, either in the same or separate packaging, and instructions for its use.

Antibodies to GLP-1 Fragments and Derivatives Thereof.

In various embodiments, monoclonal or polyclonal antibodies specific to GLP-1 fragments or derivatives thereof, can be used in immunoassays to measure the amount of GLP-1 fragments or derivatives thereof or used in immunoaffinity purification of GLP-1 fragments or derivatives thereof. A Hopp & Woods hydrophilic analysis (see Hopp & Woods, Proc. Natl. Acad. Sci. U.S.A. 78:3824-3828 (1981) can be used to identify hydrophilic regions of a protein, and to identify potential epitopes of a GLP-1 fragment or derivatives thereof.

The antibodies that immunospecifically bind to GLP-1 fragments or derivatives thereof can be produced by any method known in the art for the synthesis of antibodies, in particular, by chemical synthesis or preferably, by recombinant expression techniques. (See, e.g., U.S. Publication No. 2005/0084449, which is incorporated herein in its entirety).

Polyclonal antibodies immunospecific for GLP-1 fragments or derivatives thereof can be produced by various procedures well-known in the art. For example, GLP-1 fragments or derivatives thereof can be administered to various host animals including, but not limited to, donkeys, goats, rabbits, mice, and rats, to induce the production of sera containing polyclonal antibodies specific for GLP-1 fragments or derivatives thereof. Various adjuvants may be used to increase the immunological response, depending on the host species, including but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T Cell Hybridomas 563 681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scfv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/O1 134; International publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

### Methods of Preparing GLP-1 Fragments or Derivatives Thereof.

Any techniques known in the art can be used in purifying GLP-1 fragments or derivatives thereof, including but not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See e.g., Scopes, PROTEIN PURIFICATION, PRINCIPLES AND PRACTICE, 3rd ed., Springer (1994). During the purification, the biological activity of GLP-1 fragments or derivatives thereof may be monitored by one or more in vitro or in vivo assays. The purity of GLP-1 fragments or derivatives thereof can be assayed by any methods known in the art, such as but not limited to, gel electrophoresis. See Scopes, supra. In some embodiments, GLP-1 fragments or derivatives thereof employed in a composition of the invention can be in the range of 80 to 100 percent of the total mg protein, or at least 80%, at least 85%, at least 90%, at least 95%, or at least 98% of the total mg protein. In one embodiment, GLP-1 fragments or derivatives thereof employed in a composition of the invention is at least 99% of the total protein. In another embodiment, GLP-1 fragments or derivatives thereof are purified to apparent homogeneity, as assayed, e.g., by sodium dodecyl sulfate polyacrylamide gel electrophoresis.

Methods known in the art can be utilized to recombinantly produce GLP-1 fragments or derivatives thereof. A nucleic acid sequence encoding a GLP-1 fragment or a derivative thereof can be inserted into an expression vector for propagation and expression in host cells.

An expression construct, as used herein, refers to a nucleic acid sequence encoding a GLP-1 fragment or a derivative thereof operably associated with one or more regulatory regions that enable expression of GLP-1 fragments or derivatives thereof in an appropriate host cell. “Operably-associated” refers to an association in which the regulatory regions and the GLP-1 fragments or derivatives thereof sequence to be expressed are joined and positioned in such a way as to permit transcription, and ultimately, translation.

The regulatory regions that are necessary for transcription of GLP-1 fragments or derivatives thereof can be provided by the expression vector. A translation initiation codon (ATG) may also be provided if a GLP-1 fragment or a derivative thereof gene sequence lacking its cognate initiation codon is to be expressed. In a compatible host-construct system, cellular transcriptional factors, such as RNA polymerase, will bind to the regulatory regions on the expression construct to effect transcription of the modified GLP-1 sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to a GLP-1 fragment or a derivative thereof gene sequence or to insert a GLP-1 fragment or a derivative thereof gene sequence into the cloning site of a vector, linkers or adapters providing the appropriate compatible restriction sites may be ligated to the ends of the cDNAs by techniques well known in the art (see e.g., Wu et al., 1987, Methods in Enzymol, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA using PCR with primers containing the desired restriction enzyme site.

An expression construct comprising a GLP-1 fragment or a derivative thereof sequence operably associated with regulatory regions can be directly introduced into appropriate host cells for expression and production of a GLP-1 fragment or a derivative thereof without further cloning. See, e.g., U.S. Pat. No. 5,580,859. The expression constructs can also contain DNA sequences that facilitate integration of a GLP-1 fragment or a derivative thereof sequence into the genome of the host cell, e.g., via homologous recombination. In this instance, it is not necessary to employ an expression vector comprising a replication origin suitable for appropriate host cells in order to propagate and express GLP-1 fragments or derivatives thereof in the host cells.

A variety of expression vectors may be used, including but are not limited to, plasmids, cosmids, phage, phagemids or modified viruses. Such host-expression systems represent vehicles by which the coding sequences of a GLP-1 fragment or a derivative thereof gene may be produced and subsequently purified, but also represent cells which may, when transformed or transfected with the appropriate nucleotide coding sequences, express GLP-1 fragments or derivatives thereof in situ. These include, but are not limited to, microorganisms such as bacteria (e.g., E. coli and B. subtilis) transformed with recombinant bacteriophage DNA, plasmid DNA or cosmid DNA expression vectors containing a GLP-1 fragment or a derivative thereof coding sequences; yeast (e.g., Saccharomyces, Pichia) transformed with recombinant yeast expression vectors containing a GLP-1 fragment or a derivative thereof coding sequences; insect cell systems infected with recombinant virus expression vectors (e.g., baculovirus) containing a GLP-1 fragment or a derivative thereof coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing GLP-1 fragments or derivatives thereof coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli and eukaryotic cells are used for the expression of a recombinant GLP-1 fragment or a derivative thereof. For example, mammalian cells such as Chinese hamster ovary cells (CHO) can be used with a vector bearing promoter element from major intermediate early gene of cytomegalovirus for effective expression of a GLP-1 fragment or a derivative thereof sequence (Foecking et al., 1986, Gene 45:101; and Cockett et al., 1990, Bio/Technology 8:2).

In bacterial systems, a number of expression vectors may be advantageously selected depending upon the use intended for the GLP-1 fragment or a derivative thereof being expressed. For example, when a large quantity of a GLP-1 fragment or a derivative thereof is to be produced, for the generation of pharmaceutical compositions of a GLP-1 fragment or a derivative thereof, vectors that direct the expression of high levels of fusion protein products that are readily purified may be desirable. Such vectors include, but are not limited to, the E. coli expression vector pCR2.1 TOPO (Invitrogen); pIN vectors (Inouye & Inouye, 1985, Nucleic Acids Res. 13:3101-3109; Van Heeke & Schuster, 1989, J. Biol. Chem., 24:5503-5509) and the like. Series of vectors like pFLAG (Sigma), pMAL (NEB), and pET (Novagen) may also be used to express the foreign proteins as fusion proteins with FLAG peptide, malE-, or CBD-protein. These recombinant proteins may be directed into periplasmic space for correct folding and maturation. The fused part can be used for affinity purification of the expressed protein. Presence of cleavage sites for specific proteases like enterokinase allows one to cleave off the GLP-1 fragment or a derivative thereof. The pGEX vectors may also be used to express foreign proteins as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. A GLP-1 fragment or a derivative thereof coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, a GLP-1 fragment or a derivative thereof coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing GLP-1 fragment or a derivative thereof in infected hosts (see, e.g., Logan & Shenk, 1984, Proc. Natl. Acad. Sci. USA 8 1:355-359). Specific initiation signals may also be required for efficient translation of inserted GLP-1 fragments or derivatives thereof coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

For producing bioactive GLP-1 fragments in cells, it is important to ensure that the first amino terminal residue is not methionine but the essential histidine 7 (or, optionally an alanine, lysine, phenylalanine, tyrosine or arginine). To ensure that the recombinant peptide starts with a histidine vs. a methionine, one can add a leader sequence to the peptide construct. Appropriate design will ensure that respective leader sequence will be cleaved off in the process of protein production, thus ensuring that the final peptide starts with the desired first residue e.g. histidine or alanine.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript and post-translational modification of the gene product, e.g., glycosylation, phosphorylation, and amidation of the gene product, may be used. Such mammalian host cells include, but are not limited to, PC12, CHO, VERY, BHK, HeLa, COS, MDCK, 293, 3T3, W138, BT483, Hs578T, HTB2, BT20 and T47D, NS0 (a murine myeloma cell line that does not endogenously produce any immunoglobulin chains), CRL7O3O and HsS78Bst cells. Expression in a bacterial or yeast system can be used if post-translational modifications are found to be non-essential for a desired activity of GLP-1 fragments or derivatives thereof.

For long-term, high-yield production of properly processed GLP-1 fragments or derivatives thereof, stable expression in cells is preferred. Cell lines that stably express GLP-1 fragments or derivatives thereof may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while a GLP-1 fragment or a derivative thereof is expressed continuously.

A number of selection systems may be used, including but not limited to, antibiotic resistance (markers like Neo, which confers resistance to geneticine, or G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); and hygro, which confers resistance to hygromycin (Santerre et al., 1984, Gene 30:147). In addition, mutant cell lines including, but not limited to, tk−, hgprt− or aprt− cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of GLP-1 fragments or derivatives thereof. Modified culture conditions and media may also be used to enhance production of GLP-1 fragments or derivatives thereof. Any techniques known in the art may be applied to establish the optimal conditions for producing GLP-1 fragments or derivatives thereof.

An alternative to producing GLP-1 fragments or derivatives thereof by recombinant techniques is peptide synthesis. For example, an entire GLP-1 fragment or a derivative thereof, or a protein corresponding to a portion of a GLP-1 fragment or a derivative thereof, can be synthesized by use of a peptide synthesizer. Conventional peptide synthesis or other synthetic protocols well known in the art may be used.

Proteins having the amino acid sequence of GLP-1 fragment or a derivative thereof or a portion thereof may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support, i.e., polystyrene beads. The proteins are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting GLP-1 fragments or derivatives thereof is accomplished using conventional procedures, such as preparative HPLC using gel permeation, partition and/or ion exchange chromatography. The choice of appropriate matrices and buffers are well known in the art and so are not described in detail herein.

### EXAMPLES

### Example 1

### In Vitro Assay of GLP-1 Receptor Stimulation by GLP-1 (7-20) and a GLP-1 (7-20) Synthetic Derivative to GLP-1 Receptor in COS Cells

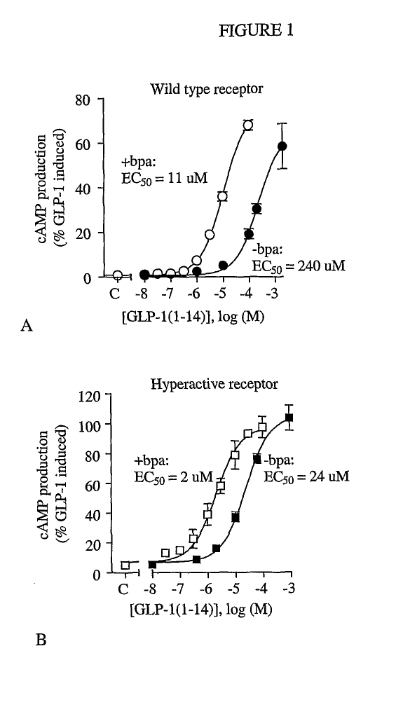

COS-7 cells were transiently transfected with cDNA encoding the wild-type human GLP-1 receptor. Peptide induced cAMP production was measured by radioimmunoassay. A 14-amino acid fragment, GLP-1 (7-20) [which as mentioned previously is the same as GLP-1 (1-14)]amide (in FIG. 1A, represented with closed circles and “−bpa”) stimulated cAMP production with a half maximal excitatory concentration (EC₅₀) of 240 μM. Carboxy-terminal tethering of a small molecule, biphenylalanine (in FIG. 1A, represented with open circles and “+bpa”) led to a 22-fold potency increase, ie. EC₅₀ of 11 μM. The data shown in FIG. 1A represent the mean±SEM of at least three experiments. This shows that GLP-1 (7-20) amide specifically interacts with the GLP-1 receptor and is able to activate cAMP production through the receptor. It also shows that the derivatization of GLP-1 (7-20) with biphenylalanine causes an increase in activation of the GLP-1 receptor.

Next, COS-7 cells were transiently transfected with cDNA encoding a hyperactive human GLP-1 receptor variant. Peptide induced cAMP production was measured by radioimmunoassay. The same peptides were used as described above, with the underivatized GLP-1 (7-20) amide represented in FIG. 1B with closed squares (“−bpa”) and the GLP-1 (7-20) carboxy-terminal tethered with a small molecule, biphenylalanine, represented by open squares (“+bpa”). GLP-1 (7-20) amide −bpa stimulated cAMP production with an EC₅₀ of 24 μM, which was a 10-fold potency increase compared to the function of this peptide at the wild type receptor. GLP-1 (7-20)+bpa had a 10-fold higher potency than this at the hyperactive receptor with an EC₅₀ of 2 μM.

Thus, while unmodified GLP-1 (7-20) amide activated cAMP production through the GLP-1 receptor, the modification of GLP-1 (7-20) increased its activity ten fold over the activity induced by the unmodified GLP-1 (7-20).

### Example 2

### Preclinical Trial Protocol for GLP-1 (7-20)

In this example, qualified animal models for diabetes are employed to examine the dose ranges of GLP-1 (7-20).

Treatment with GLP-1 (7-20) Animal Model. Zucker diabetic fatty (ZDF) rats are a well characterized type 2 diabetes animal model. (Suh Y H, et al. J Mol. Endocrinol. 2005 April; 34(2):299-315.)

Procedure: In a parental colony of ZDF rats, each rat receives dosages of GLP-1(7-20) and is compared to ZDF rats who does not receive GLP-1 (7-20) amide. Doses of GLP-1 (7-20) range from 1 μg/kg/day to 100 mg/kg/day.

Treatment. Cohorts are treated in 2 arms with 2-4 dose ranges of GLP-1 (7-20) amide and a placebo, at a compensated dose for animal size, metabolism and circulation. Arm 1: saline, Arm 2: GLP-1 (7-20) amide.

Study Assessment. Blood glucose levels are measured every week with a One Touch II glucose meter (Lifescan). Blood insulin levels are also measured. Rats are considered diabetic after 2 consecutive blood glucose measurements over 300 mg/dl. For histological analysis, pancreases are snap-frozen. Multiple 5-μm sections are stained with hematoxylin and eosin and scored blindly for islet cell mass as known in the art.

Results. ZDF rats taking GLP-1 (7-20) display a pronounced reduction in blood glucose levels and increased islet cell mass in their pancreases.

### Example 3

### Preclinical Trial Protocol for GLP-1 (7-20)−bpa and GLP-1 (7-20)+bpa

In this example, qualified animal models for diabetes are employed to examine the dose ranges of GLP-1 (7-20)−bpa and GLP-1 (7-20)+bpa.

Treatment with GLP-1 (7-20)−bpa and GLP-1 (7-20)+bpa Animal Model. Zucker diabetic fatty (ZDF) rats are a well characterized type 2 diabetes animal model. (Suh Y H, et al. J Mol. Endocrinol. 2005 April; 34(2):299-315.)

Procedure: In a parental colony of ZDF rats, each animal receives dosages of GLP(7-20)−bpa and/or GLP(7-20)+bpa and are compared to ZDF rats who receive neither. Doses of GLP(7-20)−bpa and GLP(7-20)+bpa range from 1 μg/kg/day to 100 mg/kg/day.

Treatment. Cohorts are treated in 4 arms with 24 dose ranges of each drug and a placebo, at a compensated dose for animal size, metabolism and circulation. Arm 1: saline, Arm 2: GLP(7-20)−bpa; Arm 3: GLP(7-20)+bpa; Arm 4: GLP(7-20)−bpa plus GLP(7-20)+bpa.

Study Assessment. Blood glucose levels are measured every week with a One Touch II glucose meter (Lifescan). Blood insulin levels are also measured. Rats are considered diabetic after 2 consecutive blood glucose measurements over 300 mg/dl. For histological analysis, pancreases are snap-frozen. Multiple 5-μm sections are stained with hematoxylin and eosin and scored blindly for islet cell mass as known in the art.

Results. ZDF rats taking GLP(7-20)−bpa, GLP(7-20)+bpa, or both GLP(7-20)-bpa and GLP(7-20)+bpa display a pronounced reduction in blood glucose levels and increased islet cell mass in their pancreases.

### Example 4

### Clinical Trial Protocol for GLP-1 (7-20)−bpa and GLP-1 (7-20)+bpa and Pravastatin

In this example, qualified animal models for diabetes are employed to examine the dose ranges of synergistic interaction of GLP-1 (7-20)−bpa, GLP-1 (7-20)+bpa and pravastatin.

Treatment with GLP-1 (7-20)−bpa and GLP-1 (7-20)+bpa and Pravastatin.

Animal Model. Zucker diabetic fatty (ZDF) rats are a well characterized type 2 diabetes animal model. (Suh Y H, et al. J Mol Endocrinol. 2005 April; 34(2):299-315.)

Procedure: In a parental colony of ZDF rats, each animal receives dosages of GLP-1 (7-20)−bpa and/or GLP-1 (7-20)+bpa and orpravastatin. These animals are compared to each other and ZDP rats who receive nothing. Doses of GLP-1 (7-20)−bpa and/or GLP-1 (7-20)+bpa range from 1 μg/kg/day to 100 mg/kg/day. Doses of pravastatin range from 10-40 mg/kg/day.

Treatment. Cohorts are treated in 6 arms with 2-4 dose ranges of each drug and a placebo, at a compensated dose for animal size, metabolism and circulation. Arm 1: saline, Arm 2: GLP-1 (7-20)−bpa; Arm 3: GLP-1 (7-20)+bpa; Arm 4: GLP-1 (7-20)−bpa plus pravastatin; Arm 5: GLP-1 (7-20)+bpa plus pravastatin; Arm 6: GLP-1 (7-20)−bpa plus GLP-1 (7-20)+bpa plus pravastatin.

Study Assessment. Blood glucose levels are measured every week with a One Touch II glucose meter (Lifescan). Blood insulin levels are also measured. Rats are considered diabetic after 2 consecutive measurements over 300 mg/dl. For histological analysis, pancreases are snap-frozen. Multiple 5-μm sections are stained with hematoxylin and eosin and scored blindly for islet cell mass as known in the art.

Results. ZDF rats taking GLP-1 (7-20)−bpa or a combination of GLP-1 (7-20)-bpa, GLP-1 (7-20)+bpa and pravastatin display a pronounced reduction in blood glucose levels and increased islet cell mass in their pancreases.

Although the present invention has been described in detail with reference to specific embodiments, those of skill in the art will recognize that modifications and improvements are within the scope and spirit of the invention, as set forth in the claims which follow. All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

We claim:

1. A glucagon-like peptide-1 fragment or a derivative thereof of the formula R7-R8-R9-R10-R11-R12-R13-R14-R15-R16-R17-R18-R19-R20-R21-R22-R23-R24-R25-R26-R27, wherein the glucagon-like peptide-1 fragment or a derivative thereof is between 9 and 21 amino acids in length, wherein the C terminus of the formula is truncated or not truncated, wherein
R7 is selected from a member of the group consisting of histidine, lysine, arginine, phenylalanine, tyrosine, alanine and a non-natural amino acid, R8 is selected from a member of the group consisting of alanine, glutamine, arginine, tyrosine, glycine, valine, histidine and a non-natural amino acid, R9 is selected from a member of the group consisting of glutamate, alanine, leucine, methionine, glutamine, arginine, tyrosine, histidine, aspartate and a non-natural amino acid, R10 is selected from a member of the group consisting of glycine alanine and a non-natural amino acid, R11 is selected from a member of the group consisting of threonine, alanine, glutamine, arginine, tyrosine, histidine and a non-natural amino acid, R12 is selected from a member of the group consisting of phenylalanine alanine and a non-natural amino acid, R13 is selected from a member of the group consisting of threonine, alanine and a non-natural amino acid, R14 is selected from a member of the group consisting of serine, alanine, glutamine, arginine, tyrosine, histidine and a non-natural amino acid, R15 is selected from a member of the group consisting of aspartate, alanine and a non-natural amino acid, R16 is selected from a member of the group consisting of truncated, valine, alanine, glutamine, arginine, leucine, tyrosine and a non-natural amino acid, R17 is selected from a member of the group consisting of truncated, serine, alanine, glutamine, arginine, tyrosine and a non-natural amino acid, R18 is selected from a member of the group consisting of truncated, serine, alanine, glutamine, arginine, lysine, tyrosine and a non-natural amino acid, R19 is selected from a member of the group consisting of truncated, tyrosine, alanine, glutamine, arginine, and a non-natural amino acid, R20 is selected from a member of the group consisting of truncated, leucine, alanine, glutamine, arginine, methionine, tyrosine and a non-natural amino acid, R21 is selected from a member of the group consisting of truncated, glutamate and a non-natural amino acid, R22 is selected from a member of the group consisting of truncated, glycine and a non-natural amino acid, R23 is selected from a member of the group consisting of truncated, glutamine and a non-natural amino acid, R24 is selected from a member of the group consisting of truncated, alanine and a non-natural amino acid, R25 is selected from a member of the group consisting of truncated, alanine and a non-natural amino acid, R26 is selected from a member of the group consisting of truncated, lysine and a non-natural amino acid, and R27 is selected from a member of the group consisting of truncated, glutamate and a non-natural amino acid,
and wherein when any of R16 to R27 are truncated each R group position C-terminal to the truncated R group is also truncated.

2. The glucagon-like peptide-1 fragment or the derivative thereof of claim 1, wherein the glucagon-like peptide-1 fragment or the derivative thereof comprises the amino acid sequence of SEQ ID NO:3.

3. The glucagon-like peptide-1 fragment or the derivative thereof of claim 1, further comprising one, two, or three small molecules covalently bonded to the glucagon-like peptide-1 fragment or the derivative thereof.

4. The glucagon-like peptide-1 fragment or the derivative thereof of claim 1, wherein the non-natural amino acid is selected from the group consisting of D-alanine, homoarginine, alpha-aminoisobutyric acid, diethylglycine, 1-aminocyclopentane-1-carboxylic acid, and 1-aminocyclohexane-1-carboxylic acid.

5. The glucagon-like peptide-1 fragment or the derivative thereof of claim 1, wherein the carboxy-terminal residues of the glucagon-like peptide-1 fragment or derivative thereof is amidated.

6. The glucagon-like peptide-1 fragment or the derivative thereof of claim 1, wherein the carboxy-terminal residues of the glucagon-like peptide-1 fragment or derivative thereof is non-amidated.

7. The glucagon-like peptide-1 fragment or the derivative thereof of claim 3, wherein the small molecules are selected from the group consisting of phenylalanine, 4-benzoylphenylalanine, benzylalanine, alanine-o-pentafluorphenyl, biphenylalanine, T0632, a biphenylalanine derivative and a T0632 derivative.

8. The glucagon-like peptide-1 fragment or a derivative thereof of claim 3, wherein the small molecules are bound to the carboxy terminus of the glucagon-like peptide-1 fragment or the derivative thereof.

9. The glucagon-like peptide-1 fragment or the derivative thereof of claim 1, wherein the glucagon-like peptide-1 fragment or the derivative thereof contains at least 9 amino acids.

10. The glucagon-like peptide-1 fragment or the derivative thereof of claim 1, wherein the glucagon-like peptide-1 fragment or the derivative thereof contains at most 21 amino acids.

11. The glucagon-like peptide-1 fragment or the derivative thereof of claim 1, wherein the glucagon-like peptide-1 fragment or the derivative thereof comprises an amino acid sequence selected from the group consisting of SEQ ID NO:2, 3, and 4.

12. A method of treating a member of the group consisting of glucose metabolism pathology, obesity, congestive heart failure and Alzheimer's disease in a subject in need thereof by administering to the subject a therapeutic amount of one or more glucagon-like peptide-1 fragments or derivatives thereof of the formula R7-R8-R9-R10-R1′-R12-R13-R14-R15-R16-R17-R18-R19-R20-R21-R22-R23-R24-R25-R26-R27, wherein the glucagon-like peptide-1 fragment or a derivative thereof is between 9 and 21 amino acids in length, wherein the C terminus of the formula is truncated or not truncated, wherein
R7 is selected from a member of the group consisting of histidine, lysine, arginine, phenylalanine, tyrosine, alanine and a non-natural amino acid, R8 is selected from a member of the group consisting of alanine, glutamine, arginine, tyrosine, glycine, valine, histidine and a non-natural amino acid, R9 is selected from a member of the group consisting of glutamate, alanine, leucine, methionine, glutamine, arginine, tyrosine, histidine, aspartate and a non-natural amino acid, R10 is selected from a member of the group consisting of glycine alanine and a non-natural amino acid, R11 is selected from a member of the group consisting of threonine, alanine, glutamine, arginine, tyrosine, histidine and a non-natural amino acid, R12 is selected from a member of the group consisting of phenylalanine alanine and a non-natural amino acid, R13 is selected from a member of the group consisting of threonine, alanine and a non-natural amino acid, R14 is selected from a member of the group consisting of serine, alanine, glutamine, arginine, tyrosine, histidine and a non-natural amino acid, R15 is selected from a member of the group consisting of aspartate, alanine and a non-natural amino acid, R16 is selected from a member of the group consisting of truncated, valine, alanine, glutamine, arginine, leucine, tyrosine and a non-natural amino acid, R17 is selected from a member of the group consisting of truncated, serine, alanine, glutamine, arginine, tyrosine and a non-natural amino acid, R18 is selected from a member of the group consisting of truncated, serine, alanine, glutamine, arginine, lysine, tyrosine and a non-natural amino acid, R19 is selected from a member of the group consisting of truncated, tyrosine, alanine, glutamine, arginine, tyrosine and a non-natural amino acid, R20 is selected from a member of the group consisting of truncated, leucine, alanine, glutamine, arginine, methionine, tyrosine and a non-natural amino acid, R21 is selected from a member of the group consisting of truncated, glutamate and a non-natural amino acid, R22 is selected from a member of the group consisting of truncated, glycine and a non-natural amino acid, R23 is selected from a member of the group consisting of truncated, glutamine and a non-natural amino acid, R24 is selected from a member of the group consisting of truncated, alanine and a non-natural amino acid, R25 is selected from a member of the group consisting of truncated, alanine and a non-natural amino acid, R26 is selected from a member of the group consisting of truncated, lysine and a non-natural amino acid, and R27 is selected from a member of the group consisting of truncated, glutamate and a non-natural amino acid, and
wherein when any of R16 to R27 are truncated each R group position C-terminal to the truncated R group is also truncated,
thereby treating the glucose metabolism pathology or obesity in the subject.

13. The method of claim 12, wherein the glucose metabolism pathology is selected from the group consisting of type 1 diabetes and type 2 diabetes.

14. The method of claim 12, wherein the non-natural amino acid is selected from the group consisting of D-alanine, homoarginine, alpha-aminoisobutyric acid, diethylglycine, 1-aminocyclopentane-1-carboxylic acid, and 1-aminocyclohexane-1-carboxylic acid.

15. The method of claim 12, wherein the carboxy-terminal residues in the one or more glucagon-like peptide-1 fragments or derivatives thereof are amidated.

16. The method of claim 12, wherein the carboxy-terminal residues in the one or more glucagon-like peptide-1 fragments or derivatives thereof are non-amidated.

17. The method of claim 12, wherein the one or more glucagon-like peptide-1 fragments or the derivatives thereof comprise the amino acid sequence of SEQ ID NO:3.

18. The method of claim 12, further comprising one, two, or three small molecules covalently bonded to the glucagon-like peptide-1 fragment or the derivative thereof.

19. The method of claim 18, wherein the small molecules are selected from the group consisting of phenylalanine, 4-benzoylphenylalanine, benzylalanine, alanine-o-pentafluorphenyl, biphenylalanine, T0632, a biphenylalanine derivative and a T0632 derivative.

20. The method of claim 18, wherein the small molecules are bound to the carboxy termini of the one or more glucagon-like peptide-1 fragments or derivatives thereof.

21. The method of claim 12, wherein the one or more glucagon-like peptide-1 fragments or the derivatives thereof contain at least 9 amino acids.

22. The method of claim 12, wherein the one or more glucagon-like peptide-1 fragments or the derivatives thereof contain at most 21 amino acids.

23. The method of claim 12, wherein the one or more glucagon-like peptide-1 fragments or the derivatives thereof comprise an amino acid sequence selected from the group consisting of SEQ ID NO:2, 3, and 4.

24. The method of claim 12, wherein the method further comprises the step of administering one or more members selected from the group consisting of a lipid modulating drug, an antidiabetic agent, an antidepressant, an appetite suppressant, and an anti-obesity agent.

25. The method of claim 24, wherein the lipid modulating agent is selected from a member of the group consisting of an hypolipidemic agent, an HMG-CoA reductase inhibitor, fibrate, an MTP inhibitor, and a squalene synthetase inhibitor.

26. The method of claim 24, wherein the antidiabetic agent is selected from a member of the group consisting of biguanides, sulfonyl ureas, glucosidase inhibitors, thiazolidinediones, aP2 inhibitors, PPAR agonists, SGLT2 inhibitors, insulin, an inhibitor of DPP IV, an inhibitor of neutral aminopeptidase 24.11, meglitinide, troglitazone, rosiglitazone, pioglitazone, englitazone, and darglitazone.

27. The method of claim 24, wherein the antidepressant is selected from a member of the group consisting of fluoxetine and desipramine.

28. The method of claim 24, wherein the appetite suppressant is sibutramine.

29. The method of claim 24, wherein the anti-obesity agent is selected from a member of the group consisting of orlistat and a β3 agonist.

30. The method of claim 12, wherein at least one symptom of type 1 diabetes, type 2 diabetes or latent autoimmune diabetes in adults is treated or reduced as a result of the administration of a therapeutic amount of one or more glucagon-like peptide fragments or derivatives thereof.

31. The method of claim 30, wherein the symptom is selected from a member of the group consisting of frequent urination, excessive thirst, extreme hunger, unusual weight loss, increased fatigue, irritability, blurry vision, genital itching, odd aches and pains, dry mouth, dry or itchy skin, impotence, vaginal yeast infections, poor healing of cuts and scrapes, excessive or unusual infections, hyperglycemia, loss of glycemic control, fluctuations in postprandial blood glucose, fluctuations in blood glucagon, fluctuations in blood triglycerides.

32. An antibody which selectively binds to a glucagon-like peptide-1 fragment or a derivative thereof comprising an amino acid sequence selected from a member of the group consisting of SEQ ID NOs:2, 3 and 4.

33. The antibody of claim 32, wherein the antibody is a monoclonal antibody.

34. The antibody of claim 32, wherein the antibody is a polyclonal antibody.

35. A kit for treating a patient having a glucose metabolism pathology, comprising a therapeutically effective dose of the one or more glucagon-like peptide-1 fragments or derivatives thereof of claim 1 and at least one agent selected from the group consisting of a lipid modulating drug, an antidiabetic agent, an antidepressant, an appetite suppressant, and an anti-obesity agent, either in the same or separate packaging, and instructions for its use.


SEQUENCE LISTING (4 sequences)

SEQ ID NO: 1 - protein, 37 aa, Homo sapiens
HDEFERHAEGTFTSDVSSYLEGQAAKEFIAWLVKGRG

SEQ ID NO: 2 - protein, 21 aa, Homo sapiens
HAEGTFTSDVSSYLEGQAAKE

SEQ ID NO: 3 - protein, 14 aa, Homo sapiens
HAEGTFTSDVSSYL

SEQ ID NO: 4 - protein, 8 aa, Homo sapiens
HAEGTFSD